(19) United States
(12) Patent Application Publication
(10) Pub. No.: US 2025/0017905 A1
(43) Pub. Date: Jan. 16, 2025

(54) COMPOSITION FOR INHIBITING HISTAMINE-N-METHYLTRANSFERASE

(71) Applicant: SUNTORY HOLDINGS LIMITED, Osaka-shi, Osaka (JP)

(72) Inventors: Makoto Katsube, Soraku-gun (JP); Hiroshi Watanabe, Soraku-gun (JP)

(73) Assignee: SUNTORY HOLDINGS LIMITED, Osaka-shi, Osaka (JP)

(21) Appl. No.: 18/708,426

(22) PCT Filed: Nov. 11, 2022
(86) PCT No.: PCT/JP2022/042016

§ 371 Date: May 8, 2024

(30) Foreign Application Priority Data
Nov. 17, 2021 (JP) 2021-187227

(51) Int. Cl. A61K 31/4172

(52) U.S. Cl. CPC A61K 31/4172

(57) ABSTRACT

The present invention aims to provide a composition for inhibiting histamine-N-methyltransferase. The present invention relates to a composition for inhibiting histamine-N-methyltransferase, containing L-ergothioneine or a salt thereof.

### TECHNICAL FIELD

The present invention relates to a composition for inhibiting histamine-N-methyltransferase. The present invention also relates to a method of inhibiting histamine-N-methyltransferase.

### BACKGROUND ART

Histamine is an amine synthesized from histidine, which is an amino acid. Histamine is present at high concentrations in mast cells. Histamine is also present in the lungs, gastric mucosa, brain, etc., and is responsible for physiological activity in each tissue. In the brain, histamine is involved in various physiological actions as a neurotransmitter. Reportedly, histaminergic nervous system dysfunction is associated with neuropsychiatric disorders such as attention deficit/hyperactivity disorder (ADHD) and eating disorder (Non-Patent Literature 1). Histamine-N-methyltransferase (HNMT) is a histamine metabolizing enzyme that is expressed in the brain, and it inactivates histamine by transferring a methyl group to histamine. A study using Hnmt knockout mice has reported that the brain histamine concentration was higher in the Hnmt knockout mice than in wild-type mice (Non-Patent Literature 1).

Ergothioneine is one of sulfur-containing amino acids. L-ergothioneine is present in nature. L-ergothioneine is known to have various physiological activities such as an antioxidant action. For example, according to Patent Literature 1, mice fed with golden/yellow oyster mushroom powder containing L-ergothioneine significantly reduced immobility time in a forced swimming test and a tail suspension test, compared to mice fed with regular diet.

### CITATION LIST

### Patent Literature

Patent Literature 1: JP 2014-193844 A

### Non-Patent Literature

Non-Patent Literature 1: Yoshikawa et al., International Journal of Molecular Sciences 2019, 20 (3): 737.

### SUMMARY OF INVENTION

### Technical Problem

Inhibition of histamine-N-methyltransferase can reduce a decrease in histamine concentration in the brain or maintain or increase the histamine concentration. A substance having a histamine-N-methyltransferase inhibitory activity and suitable for daily intake as a food or beverage or the like is useful for reducing a decrease in histamine concentration in the brain or maintaining or increasing the histamine concentration in the brain.

The present invention aims to provide a composition for inhibiting histamine-N-methyltransferase. The present invention also aims to provide a method of inhibiting histamine-N-methyltransferase.

### Solution to Problem

As a result of extensive studies to solve the above problems, the present inventors found that L-ergothioneine has a histamine-N-methyltransferase inhibitory action.

Specifically, the present invention relates to, but is not limited to, the following composition for inhibiting histamine-N-methyltransferase.
(1) A composition for inhibiting histamine-N-methyltransferase, containing L-ergothioneine or a salt thereof.
(2) The composition according to (1) above, wherein the composition is an oral composition.
(3) The composition according to (1) or (2) above, wherein the composition is a food or beverage.
(4) The composition according to any one of (1) to (3) above for use in preventing or ameliorating attention deficit/hyperactivity disorder or for increasing appetite.
(5) A method of inhibiting histamine-N-methyltransferase, the method including administering L-ergothioneine or a salt thereof.
(6) Use of L-ergothioneine or a salt thereof for inhibiting histamine-N-methyltransferase.

### Advantageous Effects of Invention

The present invention can provide a composition for inhibiting histamine-N-methyltransferase. The composition of the present invention can be used as a food or beverage, a pharmaceutical product, or the like. The present invention can also provide a method of inhibiting histamine-N-methyltransferase.

### BRIEF DESCRIPTION OF DRAWINGS

FIG. 1 shows evaluation results of histamine-N-methyltransferase inhibitory activity of L-ergothioneine.

### DESCRIPTION OF EMBODIMENTS

The composition of the present invention for inhibiting histamine-N-methyltransferase contains L-ergothioneine or a salt thereof. Hereinafter, the composition of the present invention for inhibiting histamine-N-methyltransferase is sometimes simply referred to as “the composition of the present invention”. The composition of the present invention usually contains L-ergothioneine or a salt thereof as an active ingredient.

L-ergothioneine is one of the amino acids.

The salt of L-ergothioneine is not limited as long as it is a pharmacologically acceptable salt or a dietary acceptable salt, and it may be either an acid salt or a basic salt. Examples of the acid salt include inorganic acid salts such as hydrochloride, sulfate, nitrate, and phosphate; and organic acid salts such as acetate, citrate, maleate, malate, oxalate, lactate, succinate, fumarate, and propionate. Examples of the basic salt include alkali metal salts such as sodium salt and potassium salt; and alkaline earth metal salts such as calcium salt and magnesium salt.

L-ergothioneine or a salt thereof that can be used may be a chemically synthesized product or a purified extract from a natural product. L-ergothioneine is abundant in golden/yellow oyster mushrooms (scientific name: Pleurotus cornucopiae var. citrinopileatus) belonging to the genus Pleurotus of the family Pleurotaceae. L-ergothioneine is also present in mushrooms such as common mushrooms (scientific name: Agaricus bisporus) including white button mushrooms, cremini mushrooms, and portabella mushrooms; grey oyster mushrooms (scientific name: Pleurotus ostreatus), shiitake (scientific name: Lentinula edodes), hen-of-the-woods (scientific name: Grifola frondosa), reishi mushurooms (scientific name: Ganoderma lucidum), lion's mane mushrooms (scientific name: Hericium erinaceus), Yanagi-matsutake (scientific name: Agrocybe aegerita), girolles (scientific name: Cantharellus cibarius), porcini mushrooms (scientific name: Boletus edulis), and common morel mushrooms (scientific name: Morchella esculenta). When L-ergothioneine is obtained from a natural product, preferably, it is extracted from a golden/yellow oyster mushroom, for example. L-ergothioneine or a salt thereof can also be produced by microbial fermentation. An extract containing L-ergothioneine or a salt thereof produced by microbial fermentation or a purified product thereof may be used. L-ergothioneine can be extracted and purified from products such as natural products by known methods. L-ergothioneine or a salt thereof may be in an isolated form.

L-ergothioneine or a salt thereof is present in natural products and food and beverages, and it is a compound that has been consumed. Thus, continuous ingestion of L-ergothioneine or a salt thereof, for example, is less likely to cause problems in terms of safety.

L-ergothioneine has a histamine-N-methyltransferase inhibitory action. The composition of the present invention is used to inhibit histamine-N-methyltransferase. L-ergothioneine or a salt thereof can be used as an active ingredient for inhibiting histamine-N-methyltransferase.

Inhibition of histamine-N-methyltransferase can reduce a decrease in histamine concentration in the brain or maintain or increase the histamine concentration in the brain. Thus, an effect can be expected which will prevent or ameliorate a condition or disease associated with a decrease in brain histamine concentration. L-ergothioneine is known to have good blood brain barrier (BBB) permeability, and it is useful for inhibiting histamine-N-methyltransferase in the brain so as to reduce a decrease in histamine concentration or maintain or increase the histamine concentration.

In one embodiment, the composition of the present invention can be used to prevent or ameliorate a condition or disease associated with a decrease in brain histamine concentration. Examples of such a condition or disease include a condition or disease accompanied by a decrease in brain histamine concentration and a condition or disease resulting from a decrease in brain histamine concentration.

Examples of the condition or disease associated with a decrease in brain histamine concentration include attention deficit/hyperactivity disorder, appetite loss, circadian rhythm disorder, decline in cognitive functions (e.g., memory, concentration, attention (including sustained attention), etc.), Parkinson's disease, Alzheimer's disease, and schizophrenia.

For example, it has been reported that the brain histamine concentration is low among patients with a condition or disease such as attention deficit/hyperactivity disorder, Parkinson's disease, or the like (Non-Patent Literature 1 described above). In addition, brain histamine is known to be involved in physiological appetite regulation (Non-Patent Literature 1 described above).

In one embodiment, the composition of the present invention can be suitably used for purposes such as preventing or ameliorating attention deficit/hyperactivity disorder and increasing appetite.

Herein, the expression “prevent a condition or disease” or its similar expression encompasses preventing onset, delaying onset, reducing the incidence rate, reducing the risk of onset, and the like. The expression “ameliorate a conditions or diseases” or its similar expression encompasses helping a subject recover from a condition or disease, alleviating a condition or disease symptoms, reducing the frequency of a condition or disease symptoms, making positive changes in a condition or disease symptoms, delaying or preventing the progression of a condition or disease, and the like.

The composition of the present invention is applicable for therapeutic use (medical use) and non-therapeutic use (non-medical use). The “non-therapeutic” is a concept that does not include medical activities, i.e., a concept that does not include methods of surgery, therapy, or diagnosis of humans.

For example, the composition of the present invention for inhibiting histamine-N-methyltransferase may be provided as an agent or the like, but it is not limited thereto. The agent can be directly provided as a composition or can be provided as a composition containing the agent. In one embodiment, the composition of the present invention for inhibiting histamine-N-methyltransferase can also be referred to as a “histamine-N-methyltransferase inhibitor”.

For a sufficient effect of the present invention, preferably, the composition of the present invention is an oral composition. The oral composition may be a food or beverage, an oral pharmaceutical product, an oral quasi-pharmaceutical product, or feed, preferably a food or beverage or an oral pharmaceutical product, more preferably a food or beverage.

The composition of the present invention can contain optional additives and optional components in addition to L-ergothioneine or a salt thereof, as long as the effect of the present invention is not impaired. Such additives and components may be selected depending on the form of the composition, for example, and those generally usable in food or beverages, pharmaceutical products, quasi-pharmaceutical products, feed, and the like can be used. When the composition of the present invention is provided as a food or beverage, a pharmaceutical product, a quasi-pharmaceutical product, feed, or the like, any common method can be used for the production.

For example, when the composition of the present invention is provided as a food or beverage, components usable in food or beverages (e.g., food materials and optional food additives) can be added to L-ergothioneine or a salt thereof to provide various food or beverages. The food or beverage is not limited. Examples thereof include general food or beverages, health foods, health drinks, foods with function claims, foods for specified health uses, health supplements, and foods for the sick. The health foods, foods with function claims, foods for specified health uses, health supplements, and the like can be used in various forms of preparations such as fine granules, tablets, granules, powders, capsules, chewable tablets, syrups, liquid agents, and liquid foods.

When the composition of the present invention is provided as a pharmaceutical product or a quasi-pharmaceutical product, for example, a pharmacologically acceptable carrier, an optional additive, or the like can be added to L-ergothioneine or a salt thereof to provide various dosage forms of pharmaceutical products or quasi-pharmaceutical products. Such a carrier, additive, or the like may be any pharmacologically acceptable one that can be used in pharmaceutical products or quasi-pharmaceutical products. Examples thereof include excipients, binders, disintegrants, lubricants, antioxidants, and colorants. One or more of these can be used. The form of administration of the pharmaceutical product or the quasi-pharmaceutical product may be oral or parenteral administration. For a sufficient effect of the present invention, oral administration is preferred. When the composition of the present invention is provided as a pharmaceutical product or a quasi-pharmaceutical product, it is preferably an oral pharmaceutical product or an oral quasi-pharmaceutical product. Examples of the dosage form for oral administration include liquids, tablets, powders, fine granules, granules, sugar-coated tablets, capsules, suspensions, emulsions, and chewable tablets. Examples of the dosage form for parenteral administration include injection and infusion.

When the composition of the present invention is provided as feed, L-ergothioneine or a salt thereof is simply added to feed. The feed includes feed additives. Examples of the feed include livestock feed for animals such as cows, pigs, chickens, sheep, and horses; feed for small animals such as rabbits, rats, and mice; and pet food for animals such as dogs, cats, and birds.

The amount of L-ergothioneine or a salt thereof in the composition of the present invention is not limited and can be set according to the composition form and the like.

For example, the amount of L-ergothioneine or a salt thereof in the composition of the present invention is preferably 0.0001 wt % or more, more preferably 0.001 wt % or more and is preferably 90 wt % or less, more preferably 50 wt % or less in terms of L-ergothioneine. In one embodiment, the amount of L-ergothioneine or a salt thereof in the composition is preferably 0.0001 to 90 wt %, more preferably 0.001 to 50 wt % in terms of L-ergothioneine. In one embodiment, when the composition of the present invention is provided as a food or beverage, a pharmaceutical product, a quasi-pharmaceutical product, feed, or the like, preferably, the amount of L-ergothioneine or a salt thereof is in the above ranges.

Herein, regarding the expression for the amount in terms of ergothioneine or an expression similar thereto, in the case of ergothioneine, the expression refers to the amount of ergothioneine; whereas in the case of a salt of ergothioneine, the expression refers to a value obtained by multiplying the molar number of the salt by the molecular weight of ergothioneine.

The composition of the present invention can be ingested or administered by an appropriate method according to the composition form. For a sufficient effect of the present invention, preferably, the composition of the present invention is orally ingested (orally administered). The intake (administration amount) of the composition of the present invention is not limited and may be any amount that achieves the histamine-N-methyltransferase inhibitory effect. The intake may be appropriately set according to the administration form, administration method, body weight of a subject, and the like.

In one embodiment, when the composition of the present invention is orally ingested by or administered to a human (adult) as a subject, the intake of L-ergothioneine or a salt thereof is preferably 2 mg or more, more preferably 5 mg or more, still more preferably 10 mg or more and is preferably 50 mg or less, more preferably 25 mg or less, still more preferably 20 mg or less in terms of L-ergothioneine per day. In one embodiment, when the composition of the present invention is orally ingested by or administered to a human (adult) as a subject, the intake of L-ergothioneine or a salt thereof is preferably 2 to 50 mg, more preferably 5 to 25 mg, still more preferably 5 to 20 mg, particularly preferably 10 to 20 mg in terms of L-ergothioneine per day. In one embodiment, when the composition of the present invention is parenterally administered to a human (adult) as a subject, the administration amount of L-ergothioneine or a salt thereof is, for example, preferably 2 to 50 mg, more preferably 5 to 25 mg, still more preferably 5 to 20 mg, particularly preferably 10 to 20 mg in terms of L-ergothioneine per day.

Preferably, L-ergothioneine or a salt thereof in the above amount is ingested or administered in one or more portions per day, for example, in one portion or several portions (e.g., two or three portions) per day. In one embodiment, in the case of a human (adult), preferably, L-ergothioneine or a salt thereof in the above amount is ingested or administered per 60 kg body weight per day. In one embodiment of the present invention, the composition of the present invention may be an oral composition for allowing L-ergothioneine or a salt thereof in the above amount to be ingested by or administered to a human per 60 kg body weight per day.

In one embodiment, the amount of L-ergothioneine or a salt thereof in the composition of the present invention is preferably 2 to 50 mg in terms of L-ergothioneine per adult daily intake. The amount of L-ergothioneine or a salt thereof in the composition of the present invention is preferably 5 to 25 mg, more preferably 5 to 20 mg, particularly preferably 10 to 20 mg in terms of L-ergothioneine per adult daily intake.

In a preferred embodiment, the composition of the present invention is continuously ingested or administered. In one embodiment of the present invention, the composition of the present invention is continuously ingested or administered preferably for one week or more, more preferably for two weeks or more. A better effect can be expected from continuous ingestion or administration of L-ergothioneine or a salt thereof.

The composition of the present invention may be ingested by or administered to any subject (administration subject). The subject is preferably a human or non-human mammal, more preferably a human. The composition of the present invention is ingested by or administered to, for example, a subject with a low brain histamine concentration, a subject needing or wanting to reduce a decrease in brain histamine concentration or maintain or increase the brain histamine concentration, a subject needing or wanting to prevent or ameliorate a condition or disease associated with a decrease in brain histamine concentration (e.g., a patient with such a condition or disease), and the like. In one embodiment, examples of the administration subject include a human with attention deficit/hyperactivity disorder and a human with a symptom of appetite loss. In one embodiment of the present invention, the administration subject of the composition of the present invention may be a healthy person. For example, the composition of the present invention may be ingested by or administered to a healthy person for a purpose of preventing a condition or disease associated with a decrease in brain histamine concentration.

The composition of the present invention may be labeled with a function claim based on inhibition of histamine-N-methyltransferase. For example, the composition of the present invention may be labeled with one or more function claims such as “not being easily distracted”, “making fewer careless mistakes”, “not being forgetful”, “being able to stay focused on one task”, “controlling overactivity of the brain”, “reducing impulsivity”, and “increasing appetite”. In one embodiment of the present invention, preferably, the composition of the present invention is a food or beverage labeled with one or more of the function claims. The label may be one indicating use of the composition for obtaining one or more of the functions. The label may be directly attached to the composition or may be attached to a container or a package of the composition.

The present invention also encompasses the following method and use:
a method of inhibiting histamine-N-methyltransferase, the method including administering L-ergothioneine or a salt thereof; and
use of L-ergothioneine or a salt thereof for inhibiting histamine-N-methyltransferase.
L-ergothioneine or a salt thereof, when ingested by or administered to a subject, can inhibit histamine-N-methyltransferase in the subject. Preferably, L-ergothioneine or a salt thereof is orally ingested or administered.

The method may be therapeutic or non-therapeutic. The use may be therapeutic or non-therapeutic.

In the method and the use, L-ergothioneine or a salt thereof, preferred embodiments thereof, and the like are the same as those of the composition of the present invention for inhibiting histamine-N-methyltransferase described above. In the method and the use, preferably, L-ergothioneine or a salt thereof is ingested by or administered to a subject at least once a day, for example, one to several times (e.g., two to three times) a day. The above use is preferably for humans or non-human mammals, more preferably for humans. In one embodiment, L-ergothioneine or a salt thereof can be used to prevent or ameliorate a condition or disease associated with a decrease in histamine concentration by inhibiting histamine-N-methyltransferase. The present invention also encompasses a method of preventing or ameliorating a condition or disease associated with a decrease in histamine concentration, the method including administering L-ergothioneine or a salt thereof.

In the methods and the use, L-ergothioneine or a salt thereof is simply used in an amount (effective amount) that provides the histamine-N-methyltransferase inhibitory effect. Preferred administration amounts of L-ergothioneine or a salt thereof, preferred administration subjects, and the like are the same as those of the composition of the present invention for inhibiting histamine-N-methyltransferase described above. L-ergothioneine or a salt thereof may be directly ingested or administered or may be ingested or administered as a composition containing L-ergothioneine or a salt thereof. For example, the composition of the present invention may be ingested or administered.

L-ergothioneine or a salt thereof can be used to produce a food or beverage, a pharmaceutical product, a quasi-pharmaceutical product, feed, or the like for use in inhibiting histamine-N-methyltransferase. In one embodiment, the present invention also encompasses use of L-ergothioneine or a salt thereof for producing a composition for inhibiting histamine-N-methyltransferase.

The present invention also encompasses L-ergothioneine or a salt thereof for use in inhibiting histamine-N-methyltransferase.

The numerical range defined by the lower limit and the upper limit herein, i.e., “the lower limit to the upper limit”, includes the lower limit and the upper limit. For example, the range defined by “1 to 2” means 1 or more and 2 or less, with 1 and 2 being inclusive. Herein, the range may be any combination of any upper limit and any lower limit.

All the academic literatures and patent literatures cited herein are incorporated herein by reference.

### EXAMPLES

The present invention is described in further detail below with reference to an example, but the scope of the present invention is not limited by the example.

### Example 1

### (Evaluation of Histamine-N-Methyltransferase Inhibitory Activity)

The study was commissioned to Eurofins Panlabs. The study was performed using recombinant human histamine-N-methyltransferase (HNMT) expressed in Escherichia coli. First, HNMT (0.025 μg/mL) was incubated with L-ergothioneine (L-ergothioneine concentration: 10 μM, 30 μM, 100 μM, 300 μM, or 1000 μM) in a phosphate buffer (pH 7.8) at 37° C. for 15 minutes. Next, 20 μM histamine, 1.4 μM S-(5′-adenosyl)-L-methionine, and 0.014 μM S-adenosyl-L-[methyl-3H]methionine were added to initiate an enzymatic reaction. After incubating at 37° C. for 30 minutes, 2.5 M borate was added to terminate the reaction. Lastly, the enzyme inhibitory activity of L-ergothioneine was evaluated using the amount of [3H]N-methylhistamine formed in the reaction as an index.

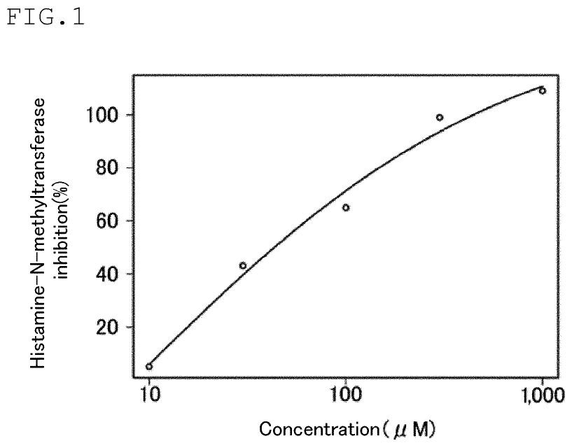

FIG. 1 shows evaluation results of the histamine-N-methyltransferase inhibitory activity of L-ergothioneine. The vertical axis (%) shows the histamine-N-methyltransferase inhibition rate. The concentration (μM) on the horizontal axis is the L-ergothioneine concentration. L-ergothioneine showed an activity to inhibit substrate binding of histamine-N-methyltransferase in a concentration-dependent manner. The L-ergothioneine concentration (IC₅₀) that inhibits 50% of substrate binding of histamine-N-methyltransferase was calculated to be 46.2 μM.

1. A composition for inhibiting histamine-N-methyltransferase, comprising
L-ergothioneine or a salt thereof.

2. The composition according to claim 1,
wherein the composition is an oral composition.

3. The composition according to claim 1,
wherein the composition is a food or beverage.

4. The composition according to claim 1 for use in preventing or ameliorating attention deficit/hyperactivity disorder or for increasing appetite.

5. A method of inhibiting histamine-N-methyltransferase, the method comprising
administering L-ergothioneine or a salt thereof.

6. Use of L-ergothioneine or a salt thereof for inhibiting histamine-N-methyltransferase.
